SEQUENCE LISTING (1 sequence)

SEQ ID NO: 1 - protein, 5 aa, Artificial Sequence
  Acetylation N-terminal
YCKPC

(19) United States
(12) Patent Application Publication
(10) Pub. No.: US 2003/0129675 A1
(43) Pub. Date: Jul. 10, 2003

(54) Assay method

(75) Inventors: Dean R. Artis, Kensington, CA (US); David Y. Jackson, San Bruno, CA (US); Susan Keating, Half Moon Bay, CA (US); Maureen Beresini, Moss Beach, CA (US)

Correspondence Address: GENENTECH, INC., 1 DNA WAY, SOUTH SAN FRANCISCO, CA, 94080, US

(73) Assignee: Genentech, Inc.

(21) Appl. No.: 10/313,541

(22) Filed: Dec. 4, 2002

Related U.S. Application Data
(63) Continuation of application No. 09/745,916, filed on Dec. 20, 2000, now abandoned.
(60) Provisional application No. 60/171,974, filed on Dec. 23, 1999.

(51) Int. Cl. G01N 33/53; G01N 33/542; C07K 7/64

(52) U.S. Cl. 435/7.9; 530/405

(57) ABSTRACT

A new method for assaying the ability of a compound to block the binding of an α₄ integrin to a binding partner thereof provides a useful screening tool.

### BACKGROUND OF THE INVENTION

1. Field of the Invention

The invention relates to a new method for assaying the ability of a compound to block the binding of an α₄ integrin to a binding ligand thereof.

2. Discussion of the Background

The migration, adhesion and subsequent extravasation of leukocytes into inflamed tissues is thought to contribute to the pathogenesis of a variety of auto inflammatory diseases including (but not limited to) asthma, rheumatoid arthritis, inflammatory bowel disease and multiple sclerosis. This process is mediated by integrin adhesion receptors expressed on the surface of the leukocytes via binding to cell adhesion molecules (CAMs) expressed at the sites of inflammation. Compounds which inhibit the interaction of the integrin receptors with their corresponding CAMs are useful anti-inflammatory agents. Current assay methods used to evaluate a compound's inhibitory activity require isolation of the CAMs as purified proteins. This invention describes the development of a general integrin binding assay useful for evaluating small molecule inhibitors for their ability to inhibit integrin/CAM interactions which does not require the use of CAMs.

### SUMMARY OF THE INVENTION

In one embodiment, the invention provides a method of detecting an inhibitor of the binding of an α₄ integrin to a binding partner thereof by combining (a) a labeled peptide (or small molecule) capable of binding an α₄ integrin and (b) a sample to be tested, with an isolated α₄ integrin under conditions suitable for binding of the isolated α₄ integrin to the labeled peptide, and detecting or measuring the amount of sample bound to the isolated α₄ integrin.

In another embodiment, the isolated α₄ integrin is α4β1 or α4β7.

In another aspect, an α₄ integrin binding protein, preferably an antibody which binds the α or β subunit of an isolated α₄ integrin, more preferably an antibody which binds the α₄ subunit of the isolated α₄ integrin, is bound to a solid support for the purpose of immobilizing the integrin. In a further embodiment, the integrin may be directly coated onto a solid phase support.

In one aspect, the labeled peptide is a cyclic peptide with a preferred formula NH2-Y—C₁—X—Z—C₂—COOH, wherein Y is an amino acid (preferably tyrosine or tyrosine analog) C₁ and C₂ are each cysteine bonded together through a disulfide bond to form a cyclic peptide, X is an amino acid linked via the side chain to a suitable label (i.e. fluorescein, biotin or other small molecule capable of binding to an antibody), and Z is an amino acid, preferably Pro, Phe, hydroxyproline, Ile, Leu, Gly, aminobenzoic acid or phenyl Gly, preferably Pro or hydroxy Pro, more preferably Pro.

In another aspect, the labeled peptide is a cyclic peptide, the cyclic peptide preferably having the formula NH₂—C₁—X₁—X₂—X₃—X₄—Y—C₂—COOH, wherein C₁ and C₂ are each cysteine bonded together through a disulfide bond to form a cyclic peptide, Y and X₁ are each independently, an amino acid, and X₂, X₃, and X₄, independently, are each a bond or an amino acid. For example, the labeled peptide may be a cyclic peptide, (1) the cyclic peptide preferably having the formula NH₂—C₁—X₁—Y—C₂—COOH, wherein C₁ and C₂ are each cysteine bonded together through a disulfide bond to form a cyclic peptide, and Y and X₁ are each independently, an amino acid; or (2) the cyclic peptide preferably having the formula NH₂—C₁—X₁—X₂—Y—C₂—COOH, wherein C₁ and C₂ are each cysteine bonded together through a disulfide bond to form a cyclic peptide, Y and X₁ are each independently, an amino acid, and X₂ is a bond or an amino acid; or (3) the cyclic peptide preferably having the formula NH₂—C₁—X₁—X₂—X₃—Y—C₂—COOH, wherein C₁ and C₂ are each cysteine bonded together through a disulfide bond to form a cyclic peptide, Y and X₁ are each independently, an amino acid, and X₂ and X₃, independently, are each a bond or an amino acid.

In each of these examples, Y may be, for example, Pro, Phe, hydroxy Pro, Ile, Leu, Gly, aminobenzoic acid or phenyl Gly, preferably Pro or hydroxy Pro, more preferably Pro. Y and X₁ may be, each independently, a naturally occurring amino acid. The label may be fluorescein isothiocyanate (FITC), biotin or any other compound capable of binding to an antibody without preventing the binding of the labeled peptide or small molecule to the integrin of interest. Scheme 1 (below) depicts a general assay format for a preferred embodiment showing the basic elements of the invention and their interaction with the other elements. In general the integrin of interest is captured on a 96-well plate using a non-blocking antibody (steps 1 and 2). Test compounds (C) premixed with the labeled peptide or small molecule (LP) are then added, followed by a label specific antibody conjugated to a suitable detection enzyme. Enzyme substrate is then added and product formation is determined spectrophotometrically.

[image]

### BRIEF DESCRIPTION OF THE FIGURE

FIGS. 1a and 1b show results of a small molecule competition assay. (a) α₄β₇ and (b) α₄β₁ were added to plates coated with anti-α₄ monoclonal antibody, at dilutions of 1/150 and 1/10 respectively. After unbound receptor was washed off, 50 ml samples of small molecules serially diluted ⅕ in Tris buffer were added to the plates, with 50 ml of 50 nM FITC labeled peptide. Bound FITC-peptide was detected by addition of anti-FITC polycolonal antibody conjugated to HRP at a 1/250 dilution. The unbound HRP conjugated antibody was washed off, followed by addition of the substrate TMB, and H₃PO₄ and the resultant OD measured at 450 nm.

### DETAILED DESCRIPTION OF THE PREFERRED EMBODIMENTS

Definitions

The term “antibody” is used in the broadest sense and specifically covers single specific polypeptides, such as monoclonal antibodies, and antibody compositions with polyepitopic specificity, i.e., “polyclonal antibodies.”

The term “biological sample” refers to a body sample from any animal, including mice, rats, dogs, monkeys and humans, but preferably is from a mammal, more preferably from a human. Such samples include biological fluids such as serum, plasma, lymph fluid, synovial fluid, follicular fluid, seminal fluid, amniotic fluid, milk, whole blood, urine, cerebrospinal fluid, saliva, sputum, tears, perspiration, mucus, and tissue culture medium, as well as tissue extracts such as homogenized tissue, and cellular extracts. The preferred biological sample herein is serum or plasma.

The term “detectable peptide” refers to a peptide, preferably a cyclic peptide, that is capable of being detected either directly through a label, which may be amplified by a detection means, or indirectly through, e.g., an antibody which binds the detectable peptide, where the antibody is labeled. For direct labeling, the peptide is typically conjugated to a moiety that is detectable by some means. The preferred detectable peptide is fluorescein isothoiocynate (FITC) or biotin labeled.

The term “detection means” refers to a moiety or technique used to detect the presence of the detectable peptide in the assay and includes detection agents that that can be used to amplify a signal correlating to the presence of an immobilized label on a microtitier plate. Preferably, the detection means is a fluorimetric, chemiluminescent, or colorimetric detection agent and may utilize avidin or streptavidin, biotin or an antibody.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al. Nature 352:624-628 (1991) and Marks et al. J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. Proc. Natl. Acad. Sci. USA 81:6851-6855 (1984)).

In general, the term “sample” means a compound or a composition containing the compound for which α4 integrin binding information is desired. The sample may be a biological sample, or other sample containing the compound. The compound may be a protein, linear or cyclic peptide, small molecule, etc.

Assay Method

The assay method of the invention is preferably a competition assay for the ability of a compound in a sample to bind an isolated α₄ integrin, such as α4β1 or α4β7 relative to a labeled peptide which has been discovered to bind to the integrin. The labeled peptides may bind to the integrin at the same site as a natural ligand for the integrin, e.g., VCAM-1 or MadCAM-1, and/or at the same site as peptides that bind anywhere on the surface of the integrin such as those generated by well known phage display techniques. See for example, U.S. Pat. No. 5,750,373; U.S. Pat. No. 5,821,047; U.S. Pat. No. 5,223,409.

First Step

In the first step of the assay herein, an isolated α₄ integrin is contacted and incubated with a capture reagent. Preferably, the α₄ integrin is immobilized with a capture (or coat) reagent which is preferably an anti-α4 or anti-β1 or -β7 monoclonal antibody or polyclonal antibody. These antibodies may be from any species, but preferably the monoclonal antibody is a murine or rat monoclonal antibody, more preferably murine. Furthermore, the antibodies are preferably affinity purified, to decrease background. In a specific preferred embodiment, the immobilized monoclonal antibody is a murine monoclonal antibody, more preferably anti-hu-α₄ (e.g., 9F10, anti-CD49d, cat#31470D, Pharmingen, San Diego, Calif.). Immobilization conventionally is accomplished by insolubilizing the capture reagent either before the assay procedure, as by adsorption to a water-insoluble matrix or surface (U.S. Pat. No. 3,720,760) or non-covalent or covalent coupling (for example, using glutaraldehyde or carbodiimide cross-linking, with or without prior activation of the support with, e.g., nitric acid and a reducing agent as described in U.S. Pat. No. 3,645,852 or in Rotmans et al. J. Immunol. Methods 57:87-98 (1983)), or afterward, e.g., by immunoprecipitation.

The solid phase used for immobilization may be any inert support or carrier that is essentially water insoluble and useful in immunometric assays, including supports in the form of, e.g., surfaces, particles, porous matrices, etc. Examples of commonly used supports include small sheets, Sephadex, polyvinyl chloride, plastic beads, and assay plates or test tubes manufactured from polyethylene, polypropylene, polystyrene, and the like including 96-well microtiter plates, as well as particulate materials such as filter paper, agarose, cross-linked dextran, and other polysaccharides. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are suitably employed for capture reagent immobilization. In a preferred embodiment the immobilized capture reagent is coated on a microtiter plate, and in particular the preferred solid phase used is a multi-well microtiter plate that can be used to analyze multiple samples at one time. The most preferred is a microtest 96-well ELISA plate such as that sold as Nunc Maxisorp or Immulon.

The solid phase is coated with the capture reagent as defined above, which may be linked by a non-covalent or covalent interaction or physical linkage as desired. Techniques for attachment include those described in U.S. Pat. No. 4,376,110 and the references cited therein. If covalent, the plate or other solid phase is incubated with a cross-linking agent together with the capture reagent under conditions well known in the art, such as for 1 hour at room temperature.

Commonly used cross-linking agents for attaching the capture reagent to the solid phase substrate include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates capable of forming cross-links in the presence of light.

If 96-well plates are utilized, they are preferably coated with the capture reagent (typically diluted in a buffer such as phosphate buffered saline (PBS)) by incubation for at least about 10 hours, more preferably at least overnight, at temperatures of about 4-20 C., more preferably about 4-8 C., and at a pH of about 6-8, more preferably about 6.5-7.5, and most preferably 7.2-7.4. If shorter coating times (1-2 hours) are desired, one can use 96-well plates with nitrocellulose filter bottoms (Millipore MULTISCREEN™) or coat at 37 C. The plates may be stacked and coated long in advance of the assay itself, and then the assay can be carried out simultaneously on multiple samples in a manual, semi-automatic, or automatic fashion, such as by using robotics.

After removing excess coating reagent, the coated plates are then typically treated with a dilute solution of a blocking agent that binds non-specifically to and saturates the binding sites on the plate surface to prevent unwanted binding of the free ligand to the excess sites on the wells of the plate, according to known methods. Examples of appropriate blocking agents for this purpose include, e.g., gelatin, bovine serum albumin, egg albumin, casein, and non-fat milk. The blocking treatment typically takes place under conditions of ambient temperatures for about 1-4 hours, preferably about 1.5 to 3 hours.

After coating and blocking, excess blocking reagent is removed, preferably by washing. The solution used for washing is generally a buffer (“washing buffer”) with a pH determined using the considerations and buffers described below for the incubation step, with a preferable pH range of about 6-9. The washing may be done 1, 2, 3 or more times. The temperature of washing is generally from refrigerator to moderate temperatures, with a constant temperature maintained during the assay period, typically from about 0-40 C., more preferably about 4-30 C. An unbound or purified α4 integrin, preferably α4β1 or α_{4β7,} appropriately diluted, is added to the immobilized phase. The preferred dilution rate is about 0.2-20%, preferably about 1.0%, by volume. Buffers that may be used for dilution for this purpose include (a) 0.05M Tris-HCl, pH 7.5, containing 0.5% BSA, 0.05% TWEEN 20™ detergent (P20), 1 mM MnCl₂, and 0.15M NaCl; (b) 0.05M Hepes, pH 7.5, containing 0.5% BSA, 0.05% TWEEN 20™ detergent (P20), 1 mM MgCl₂, 1 mM CaCl₂, and 0.15M NaCl; (c) 0.05M Tris-HCl, pH 7.5, containing 0.5% bovine gamma globulin, 0.05% TWEEN 20™ detergent (P20), 1 mM MnCl₂, and 0.15M NaCl; (d) 0.05M Hepes, pH 7.5, containing 0.5% bovine gamma globulin, 0.05% TWEEN 20™ detergent (P20), 1 mM MgCl₂, 1 mM CaCl₂, and 0.15M NaCl; (e) 0.05M Tris-HCl, pH 7.5, containing 0.05% TWEEN 20™ detergent (P20), 1 mM MnCl₂, and 0.15 M NaCl; (d) 0.05M Hepes, pH 7.5, containing 0.05% TWEEN 20™ detergent (P20), 1 mM MgCl₂, 1 mM CaCl₂, and 0.15M NaCl. Buffer (a) is the preferred buffer for the assay herein since it has the best differentiation between each standard as well as the biggest signal-to-noise ratio. TWEEN 20™ acts as a detergent to eliminate non-specific binding.

The conditions for incubation are selected to maximize capture of the integrin by the antibody and minimize dissociation. Preferably, the incubation is accomplished at fairly constant temperatures, ranging from ambient temperature to about 40 C., preferably from about 36 to 38 C. to obtain a less variable, lower coefficient of variant (CV) than at, e.g., room temperature. The time for incubation depends primarily on the temperature, being generally no greater than about 10 hours. Preferably, the incubation time is from about 0.5 to 3 hours, and more preferably 1.5-3 hours at 36-38 C. to maximize binding of free to capture reagents.

At this stage, the pH of the incubation mixture will ordinarily be in the range of about 6-9.5, preferably in the range of about 7-8, and most preferably the pH of the assay diluent is 7.5±0.1. Various buffers may be employed to achieve and maintain the desired pH during this step, including borate, phosphate, carbonate, Tris-HCl or Tris-phosphate, Hepes, acetate, barbital, and the like. The particular buffer employed is not critical to the invention, but in individual assays one buffer may be preferred over another.

Second Step

In a second step of the assay method herein, the unbound (purified) integrin is separated (preferably by washing as described above) from the immobilized capture reagent to remove uncaptured integrin.

Third Step

After washing, the immobilized capture reagent is contacted with a sample and a labeled peptide, in order to allow competitive binding of the sample and labeled peptide to the immobilized integrin, and incubated. The conditions for incubation are selected to maximize competitive binding and minimize dissociation. Time, temperature and pH conditions may be generally those discussed above. Washing is conducted as described above in Step 2.

Fourth Step

If the labeled peptide is directly detectable, this step is optional and one may proceed to the Fifth Step. In this step, after optional washing as described above, the immobilized capture reagent/integrin/labeled peptide complex is contacted with a detectable molecule, for example a protein or is peptide binding partner for the labeled peptide, preferably an antibody or strepavidin, and preferably at a temperature of about 20-40 C., more preferably about 36-38 C., with the exact temperature and time for contacting the two being dependent primarily on the detection means employed. For example, when 4-methylumbelliferyl-β-galactoside (MUG) and streptavidin—galactosidase are used as the means for detection, preferably the contacting is carried out overnight (e.g., about 15-17 hours or more) to amplify the signal to the maximum. The detectable molecule may be a polyclonal or monoclonal antibody or strepavidin. Also, the detectable antibody may be directly detectable, and preferably has a fluorimetric label. The fluorimetric label has greater sensitivity to the assay compared to a conventional colorimetric label. The detectable antibody can be biotinylated and the detection means is avidin or streptavidin—galactosidase and MUG. Alternatively, the detectable molecule (e.g., peptide, protein, antibody) may be conjugated to an enzyme and detection accomplished by monitoring the absorbance or fluorescence of an enzymatic product following the addition of a suitable substrate for the enzyme, using well known enzyme detection systems such as alkaline phosphatase or horse radish peroxidase (Anti-fluorescein-HRP or -AP (cat#NEF710 and NEF709, Dupont NEN, Boston, Mass.).

Preferably a molar excess of a detectable molecule with respect to the maximum concentration of labeled peptide (as described above) is added to the plate after it is washed. This detectable molecule (which is directly or indirectly detectable) is preferably a polyclonal antibody, although any antibody can be employed. The affinity of the antibody must be sufficiently high that small amounts of the labeled peptide can be detected, but not so high that it causes the labeled peptide to be pulled from the capture reagent/integrin.

Fifth Step

In the last step of the assay method, the level of labeled peptide that is now bound to the capture reagent/integrin is measured using a detection means for the directly detectable label of the labeled peptide or the detectable molecule. If desired, the measuring step may comprise comparing the reaction that occurs as a result of the above described steps with a standard curve to determine the level of relative binding compared to an optional standard.

Antibody Production

Polyclonal antibodies generally are raised in animals by multiple subcutaneous (sc) or intraperitoneal (ip) injections of an integrin and an adjuvant. It may be useful to conjugate the integrin or a fragment containing the target amino acid sequence to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

The antibodies used as the coat or detectable molecules may be obtained from any convenient vertebrate source, such as murine, primate, lagomorpha, goat, rabbit, rat, chicken, bovine, ovine, equine, canine, feline, or porcine. Chimeric or humanized antibodies may also be employed, as described, e.g., in U.S. Pat. No. 4,816,567; Morrison et al. Proc. Natl. Acad. Sci. USA 81:6851 (1984); Neuberger et al. Nature 312: 604 (1984); Takeda et al. Nature 314:452 (1985); and WO 98/45331 published Oct. 15, 1998, as well as in those additional references set forth above.

Animals may be immunized against the immunogenic conjugates or derivatives by combining 1 mg or 1 μg of conjugate (for rabbits or mice, respectively) with 3 volumes of Freund's complete adjuvant and injecting the solution intradermally at multiple sites. One month later the animals are boosted with ⅕ to 1/10 the original amount of conjugate in Freund's incomplete adjuvant by subcutaneous injection at multiple sites. 7 to 14 days later animals are bled and the serum is assayed for antibody titer. Animals are boosted until the titer plateaus. Preferably, the animal is boosted with the conjugated integrin, but conjugated to a different protein and/or through a different cross-linking agent. Conjugates also can be made in recombinant cell culture as protein fusions. Also, aggregating agents such as alum are used to enhance the immune response. Methods for the production of polyclonal antibodies are described in numerous immunology textbooks, such as Davis et al. Microbiology, 3rd Edition, (Harper & Row, New York, N.Y., 1980).

Monoclonal antibodies are prepared by recovering spleen cells from immunized animals and immortalizing the cells in conventional fashion, e.g. by fusion with myeloma cells or by Epstein-Barr virus transformation, and screening for clones expressing the desired antibody. See, e.g., Kohler and Milstein Eur. J. Immunol. 6:511 (1976). Monoclonal antibodies, or the antigen-binding region of a monoclonal antibody, such as Fab or (Fab)₂ fragments, may alternatively be produced by recombinant methods.

Examples of suitable antibodies include those already utilized in known assays for the integrins in question, e.g., those antibodies directed against the integrin which are well known in the art and are non-function blocking, that is, a suitable antibody will not block binding of the labeled peptide to the integrin.

Detection

The labeled peptide added to the immobilized capture reagent/integrin complex may be either directly detected by way of a directly detectable label on the labeled peptide, or detected indirectly by addition of a molar excess of a detectable molecule, for example a detectable labeled antibody directed against the label of the labeled peptide.

The label used for the labeled peptide or the detectable molecule may be any detectable functionality that does not interfere with the binding of the integrin to the labeled peptide or binding of the labeled peptide to the detectable molecule. Examples of suitable labels are those numerous labels known for use in immunoassay, including moieties that may be detected directly, such as fluorochrome, chemiluminscent, and radioactive labels, as well as moieties, such as enzymes, that must be reacted or derivatized to be detected. Examples of such labels include the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, biotin/streptavidin, biotin/Streptavidin-β-galactosidase with MUG, streptavidin-hydrogen peroxidase, spin labels, bacteriophage labels, stable free radicals, and the like. Detection with an enzyme labeled detectable antibody as the detectable molecule is preferred.

Conventional methods are available to bind these labels covalently to proteins or polypeptides. For instance, coupling agents such as dialdehydes, carboduimides, dimaleimides, bis-imidates, bis-diazotized benzidine, and the like may be used to tag the antibodies with the above-described fluorescent, chemiluminescent, and enzyme labels. See, for example, U.S. Pat. Nos. 3,940,475 (fluorimetry) and 3,645,090 (enzymes); Hunter et al. Nature 144:945 (1962); David et al. Biochemistry 13:1014-1021 (1974); Pain et al. J. Immunol. Methods 40:219-230 (1981); and Nygren J. Histochem. and Cytochem. 30:407-412 (1982). Labels may be fluorescent and chemiluminescent to increase amplification and sensitivity, more preferably antibody or strepavidin with horse radish peroxidase and tetramethyl benzidine for amplifying the signal.

The conjugation of such label, including the enzymes, to the labeled peptide or to the detectable molecule is a standard manipulative procedure for one of ordinary skill in immunoassay techniques. See, for example, O'Sullivan et al. “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166.

Following the addition of last reagent, the amount of bound labeled peptide is determined by removing excess unbound reagent, or detectable molecule through washing and then measuring the amount of the attached label using a detection method appropriate to the label, and correlating the measured amount with a standard. For example, in the case of enzymes, the amount of color developed and measured will be a direct measurement of the amount of labeled peptide present. Specifically, if HRP is the label, the color is detected using the substrate TMB (3,3′,5,5′-tetramethylbenzidine Peroxidase Substrate System, Kirkegaard & Perry Laboratories, Inc., Gaithersburg, Md.) at 450 nm absorbance, sometimes after addition of a stop reagent, for example, 1M H₃PO₄. Other known HRP substrates may be used.

In one example, after an enzyme-labeled antibody directed against the labeled peptide is washed from the immobilized phase, color or luminescence is developed and measured by incubating the immobilized capture reagent with a substrate of the enzyme. Then the amount of bound labeled peptide is calculated by comparing with the color or luminescence generated by the standard run in parallel.

Peptides

Peptides for use as the labeled peptide in the method of the invention are preferably cyclic peptides having the formula NH₂—C₁—X₁—X₂—X₃—X₄—Y—C₂—COOH, where C₁ and C₂ are each cysteine bonded together through a disulfide bond to form a cyclic peptide, Y and X₁ are each independently, an amino acid, and X₂, X₃, and X₄, independently, are each a bond or an amino acid. In one specific embodiment, the cyclic peptide had the formula NH₂—C₁—X₁—Y—C₂—COOH, where C₁ and C₂ are each cysteine bonded together through a disulfide bond to form a cyclic peptide, and Y and X₁ are each independently, an amino acid. In another embodiment, the cyclic peptide has the formula NH₂—C₁—X₁—X₂—Y—C₂—COOH, where C₁ and C₂ are each cysteine bonded together through a disulfide bond to form a cyclic peptide, Y and X₁ are each independently, an amino acid, and X₂ is a bond or an amino acid. In another embodiment, the cyclic peptide preferably having the formula NH₂—C₁—X₁—X₂—X₃—Y—C₂—COOH, where C₁ and C₂ are each cysteine bonded together through a disulfide bond to form a cyclic peptide, Y and X₁ are each independently, an amino acid, and X₂ and X₃, independently, are each a bond or an amino acid.

Y is Pro, Phe, hydroxy Pro, Ile, Leu, Gly, aminobenzoic acid or phenyl Gly, preferably Pro or hydroxy Pro, more preferably Pro.

Specific examples of suitable cyclic peptides include C-K-P-C; Y-C-Ornithine-P-C and Y-C-diaminopropionic acid-P-C.

The cyclic peptides may be synthesized using methods generally described and known in the field of synthetic peptide chemistry. See for example Jackson, D. Y. et al, 1997, J. Med. Chem., 40:3359-3368 as well as the description in Examples 1 and 2 and in Scheme 2.

The following examples are intended to illustrate one embodiment now known for practicing the invention, but the invention is not to be considered limited to these examples. All open and patented literature citations herein are expressly incorporated by reference.

### EXAMPLES

### Example 1

Synthesis of Cyclic Peptide (1).

The cyclic peptide Ac-YCKPC (1) was synthesized as previously described (Jackson, D. Y. et al, 1997, J. Med. Chem., 40:3359-3368) using standard solid phase peptide chemistry (Merrifield, R. B., 1963, J. Am. Chem. Soc. 85:2149-2154) with FMOC protected amino acids (Carpino, L. A., et al, 1972, J. Org. Chem. 37:3404-3409) on a p-alkoxybenzyl alcohol resin (Wang, S. S., et al, 1978, Int. J. Peptide Protein Research 11:297-299). Amino acids were purchased from Advanced ChemTech U.S.A. Couplings were performed with 4 eq. of HBTU activated amino acid and 4 eq. of N-methylmorpholine. FMOC groups were removed with 20% piperidine in DMA. Cleavage and deprotection with TFA containing 5% triethylsilane afforded the crude linear peptide Ac-YCKPC. The crude peptide was then extracted from the resin with 100 mL of 2:1 H₂O/CH₃CN. Disulfide oxidation was carried out at 25 C via drop wise addition of a saturated solution of iodine in acetic acid to the crude extracts with vigorous stirring until a slight yellow color persisted. The crude oxidized peptide was lyophilized and purified by preparative reverse phase C18 HPLC (CH₃CN/H₂O gradient, 0.1% TFA). Pure fractions (>98% pure by analytical HPLC) were combined, lyophilized and characterized by electrospray ionization mass spectrometry (MH+ calc.=653.8; found 654.0).

### Example 2

Synthesis of Fluorescein Labeled Peptide (3).

The fluorescein labeled cyclic peptide inhibitor (3, (B)-cyclo-CK(FITC)PC-CO₂H, where (B) is N-acetyl-3-(4-hydroxyphenyl)-proline and FITC is fluorescein isothiocyanate) was synthesized as follows (Scheme 2). Compound (1) from Example 1 above (100 mg) was dissolved in 3 mL of DMF and 100 μL of DIPEA was added followed by 100 mg of fluorescein isothiocyanate (FITC, Sigma). After stirring at 250 C. for 4 h, the mixture was poured into 20 mL of H2O and acidified with 200 μL of acetic acid. The labeled peptide was extracted from the acidified solution with 50 mL of ethyl acetate to afford 75 mg of crude (3) after evaporation of solvent. Purification by preparative reverse phase C18 HPLC (CH₃CN/H₂O gradient, 0.1% TFA) afforded 42 mg of pure (3) as determined by analytical HPLC and MS analysis (MH+ calc.=1069.2; found 1070.0) suitable for use in assays.

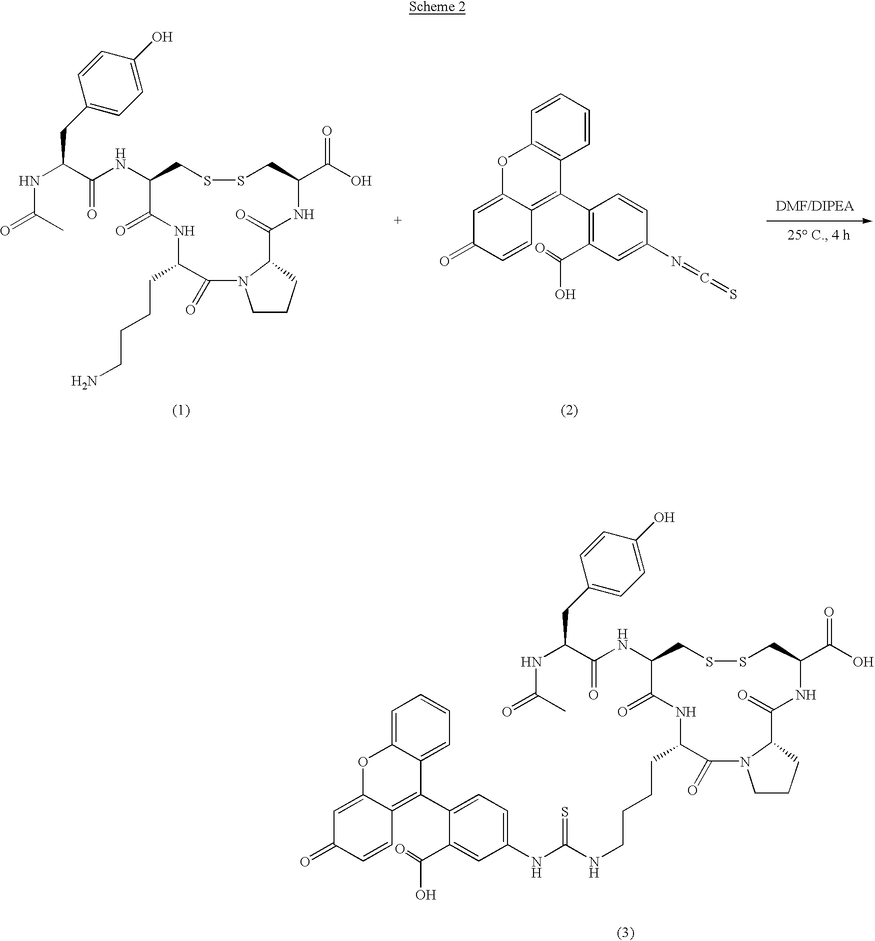

### Example 3

Competition ELISA Using the Fluorescein Labeled Peptide (3) for Determining Small Molecule Binding Affinities for α₄β₇ and/or α₄β₁.

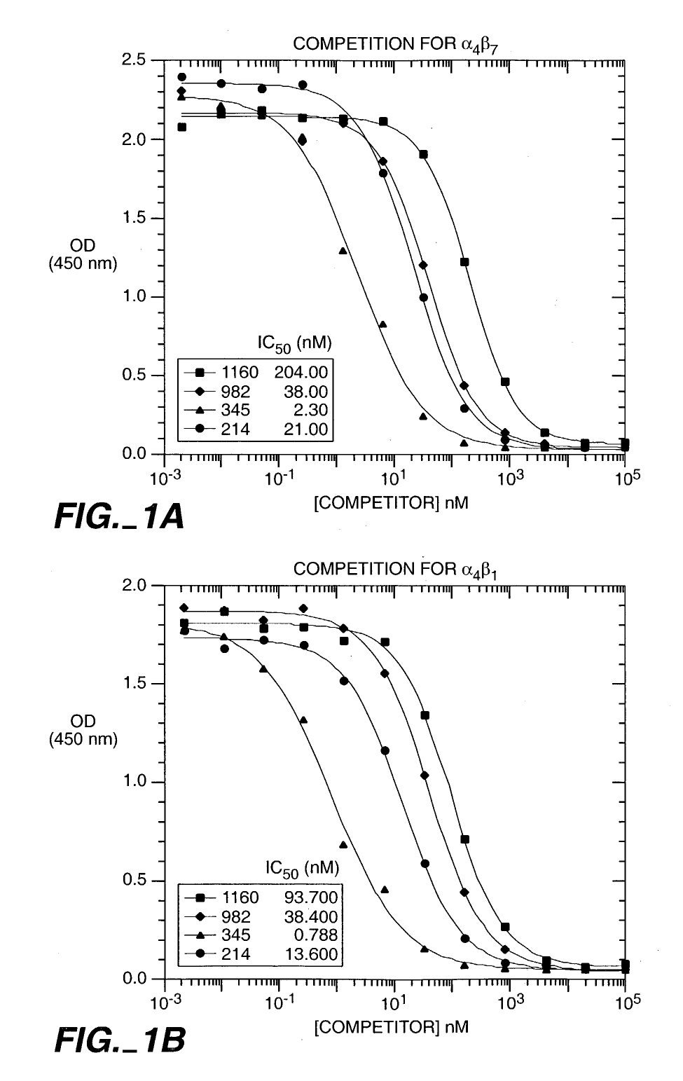

Compounds were assayed for their ability to bind α₄β₇ and α₄β₁ in a competition format ELISA (enzyme linked immunosorbent assay) as follows. 96-well plates were coated with mouse anti-human α₄ Ig or mouse anti-human β₁ Ig in an appropriate buffer (PBS or other) for 4-12 hours at room temperature, washed and blocked with 0.5% BSA in PBS for 1 hour. After washing the plates to remove BSA, α₄β₇ or α₄β₁ (10-100 ng/mL) in an appropriate buffer was added, incubated for 2 hours at room temperature and the plates washed again to remove excess receptor. Serial dilutions (5×) of the test compounds (1 nM-100 μM) in an appropriate buffer (phosphate buffered saline, PBS) were mixed with the fluorescein labeled peptide (3) (1 μM in PBS), added to the plates and incubated for 1-2 hours. After washing with PBS, a solution of sheep anti-FITC/horseradish peroxidase (HRP) conjugate (Sigma) or sheep anti-FITC/AP (alkaline phosphatese) was added and the plates incubate an additional hour at room temperature. After washing to remove unbound conjugate, an appropriate enzyme substrate is added (tetramethylbenzidine for HRP or 2,4-dinitrophenyl phosphate for AP) and incubated for 30-60 minutes until a sufficient color intensity is achieved (˜1-2 OD). Spectrophotometric measurement of the color intensity is used to quantitate the amount of fluorescein peptide bound. The relative affinities of test compounds for α₄β₇ or α₄β₁ are determined by plotting the absorbance versus the concentration of inhibitor; the concentration of inhibitor correlated with half maximal absorbance is reported as the IC₅₀. The IC₅₀s determined for several representative α4 inhibitors is shown in Table 1. See also FIG. 1. For comparative purposes, the IC₅₀s obtained using a protein based ELISA are also shown in Table 1.

TABLE 1
Comparison of IC₅₀s obtained using the fluorescent peptide
ELISA with those obtained using a protein ELISA

IC₅₀ (nM)

| | α4β7/MAdCAM | α4β7/ (3) |
|---|---|---|
| G # | (protein ELISA*) | (labeled peptide ELISA) |
| 016244 | 4.2 | 8.9 |
| 016390 | 3.3 | 5.0 |
| 016426 | 5.1 | 18.0 |
| 016617 | 10.0 | 29.0 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method of detecting an inhibitor of the binding of an α₄ integrin to a binding partner thereof, comprising
combining (a) a labeled peptide capable of binding an α₄ integrin and (b) a sample to be tested, with an isolated α₄ integrin under conditions suitable for binding of the isolated α₄ integrin to the labeled peptide, and
detecting or measuring the amount of sample bound to the isolated α₄ integrin.

2. The method of claim 1, wherein the isolated α₄ integrin is α4β1 or α4β7.

3. The method of claim 1, wherein the isolated α₄ integrin is bound to a solid support.

4. The method of claim 1, wherein an α₄ integrin binding protein, preferably an antibody which binds the α or β of the isolated α₄ integrin, more preferably an antibody which binds the α₄ subunit of the isolated α₄ integrin, is bound to a solid support.

5. The method of claim 1, wherein the labeled peptide is a cyclic peptide, the cyclic peptide preferably having the formula NH₂—C₁—X₁—X₂—X₃—X₄—Y—C₂—COOH, wherein C₁ and C₂ are each cysteine bonded together through a disulfide bond to form a cyclic peptide, Y and X₁ are each independently, an amino acid, and X₂, X₃, and X₄, independently, are each a bond or an amino acid.

6. The method of claim 1, wherein the labeled peptide is a cyclic peptide, the cyclic peptide preferably having the formula NH₂—C₁—X₁—Y—C₂—COOH, wherein C₁ and C₂ are each cysteine bonded together through a disulfide bond to form a cyclic peptide, and Y and X₁ are each independently, an amino acid.

7. The method of claim 1, wherein the labeled peptide is a cyclic peptide, the cyclic peptide preferably having the formula NH₂—C₁—X₁—X₂—Y—C₂—COOH, wherein C₁ and C₂ are each cysteine bonded together through a disulfide bond to form a cyclic peptide, Y and X₁ are each independently, an amino acid, and X₂ is a bond or an amino acid.

8. The method of claim 1, wherein the labeled peptide is a cyclic peptide, the cyclic peptide preferably having the formula NH₂—C₁—X₁—X₂—X₃—Y—C₂—COOH, wherein C₁ and C₂ are each cysteine bonded together through a disulfide bond to form a cyclic peptide, Y and X₁ are each independently, an amino acid, and X₂ and X₃, independently, are each a bond or an amino acid.

9. The method of claim 5, wherein Y is Pro, Phe, hydroxy Pro, Ile, Leu, Gly, aminobenzoic acid or phenyl Gly, preferably Pro or hydroxy Pro, more preferably Pro.

10. The method of claim 5, wherein Y and X₁ are each independently, a naturally occurring amino acid.

11. The method of claim 1, wherein the label is fluorescein isothiocyanate.
